(19) United States
(12) Patent Application Publication
(10) Pub. No.: US 2008/0248463 A1
(43) Pub. Date: Oct. 9, 2008

(54) Split Enzyme Linked Immunosorbent and Nucleic Acid Assays

(75) Inventors: Shimon Weiss, Los Angeles, CA (US); Anna M. Wu, Los Angeles, CA (US); L. Jean Perry, Los Angeles, CA (US)

Correspondence Address: VENABLE LLP, P.O. BOX 34385, WASHINGTON, DC, 20043-9998, US

(73) Assignee: The Regents of the University of California, Oakland, CA (US)

(21) Appl. No.: 10/593,059

(22) PCT Filed: Mar. 14, 2005
(86) PCT No.: PCT/US05/08621

§ 371 Date: Sep. 15, 2006

Related U.S. Application Data
(60) Provisional application No. 60/553,579, filed on Mar. 15, 2004.

(51) Int. Cl. C12Q 1/68; G01N 33/573; G01N 33/567; C12Q 1/02; C40B 30/04

(52) U.S. Cl. 435/6; 435/7.4; 435/7.2; 435/29; 506/9

(57) ABSTRACT

A method for detecting the presence of an analyte in a solution. The analyte includes at least two mutually exclusive recognition sites that are capable of binding to corresponding recognition molecules. Biosensors are provided that include the recognition molecules, which are attached to the inactive portions of a split enzyme. The recognition sites are located such that the inactive enzyme portions combine to form a detectable biologically active enzyme when the recognition molecules bind to recognition sites.

This invention was made with Government support of Grant No. DE-FG03-02ER63339, awarded by DOE. The Government has certain rights in this invention.

### BACKGROUND OF THE INVENTION

1. Field of the Invention

The present invention relates generally to methods that are based on detecting the presence of one or more analytes in a solution. More particularly, the present invention involves splitting a biologically active enzyme into at least two portions that have reduced biologically activity. The two inactive enzymes are then linked to targeting or recognition molecules that target the enzyme portions to mutually exclusive recognition sites on an analyte. Upon reaching the recognition sites, the enzyme portions interact to form the biologically active enzyme, which can be detected using conventional enzyme detection techniques.

2. Description of Related Art

There is a great need for high specificity, high sensitively in vitro detection and quantification methods for analytes derived from cell lysates and/or secreted molecules (proteins, antigens and pathogens) in proteomics, clinical diagnosis and pharmacogenomics. An analogous need exists for detection of pathogens in the environment (bio-terrorism), in food (product safety control), etc. In short, there is a need for a solution based, “PCR equivalent” for non nucleic-acid analytes.

Furthermore, PCR itself for detection of nucleic acid sequences in analytes is complicated and expensive. For example, expression analysis and detection of particular sequences in genomic samples require two enzymatic reactions: RT and PCR (as in DNA arrays). A cheaper and simpler nucleic acid-based detection and quantification assay will have high utility.

Lastly, there are no methods to quantify mRNA and translated protein levels in the live cell, in real time. Such live cell based assays will be very powerful for drug discovery, bioinformatics and pharmacogenomics.

### SUMMARY OF THE INVENTION

In accordance with the present invention, a method is provided for detecting the presence of an analyte in a solution where the analyte includes at least two mutually exclusive recognition sites that are capable of binding to corresponding mutually exclusive recognition molecules. The invention utilizes split enzyme biosensors, which include the recognition molecules that bind to the recognition sites on the analyte.

As a feature of the present invention, the biosensors are composed of at least a first sensor and a second sensor. The first sensor includes a first recognition molecule that is capable of binding to one of the recognition sites on the analyte. The first sensor also includes a first enzyme portion that is attached to the first recognition molecule. The second sensor includes a second recognition molecule that is capable of binding to another of the recognition sites on the analyte. The second sensor also includes a second enzyme portion that is attached to the second recognition molecule.

In order to detect the presence of the analyte, the first sensor and second sensor are mixed with a solution that is suspected of containing the analyte. The recognition sites on said analyte include at least a first recognition site and a second recognition site. The first recognition site is capable of binding only to the first sensor and the second recognition site is capable of binding only to the second sensor. As a feature of the present invention, the first recognition site and second recognition site are located within the analyte such that the first enzyme portion and second enzyme portion of the sensors combine to form a biologically active enzyme when the first and second recognition molecules bind to the first and second recognition sites. The term “biologically active” is used in its normal sense to mean that the enzyme exhibits its normal activity even though the analysis may be conducted in vitro.

The presence of the analyte is detected by determining if the biologically active enzyme has been formed. The step of detecting if the biologically active enzyme has been formed is carried out using known enzyme detection techniques (cleavage, ligation, colorometric, bioluminescence, fluorescence and conformational change) where the biologically active enzyme is reacted with a first reactant to form a first product that can be detected by spectrophotometry, fluoroscopy and the like.

As a further feature of the invention, the gain of the method may be enhanced by forming allosteric enzyme activators (any coenzyme) as the first product. The first reactant is chosen such that the result of its interaction with the biologically active enzyme is the formation of an allosteric activator, which is capable of activating a second enzyme that has been added to the solution. The allosteric activator reacts with the second enzyme to form an activated amplifier enzyme. The activated amplifier enzyme is then reacted with a second reactant in the solution to form a second amplified product, which is detected by spectrophotometry, fluoroscopy and the like.

In addition to their use in methods for detecting analytes, the biosensors of the present invention may also be used to form logic AND gates, logic OR gates, polymerase chain reaction (PCR) procedures, molecular evolution and single molecule DNA sequencing. The biosensors may also be used to form an enzymatic factory where multiple pairs of recognition sites are located along DNA or another macromolecule. Biologically active enzymes are formed sequentially at the multiple pairs of recognition sites along the DNA. As a feature of this system, the product of the biologically active enzyme at the first site is capable of reacting with the biologically active enzyme at the second site to form a second product which, in turn, is capable of reacting with the next sequential biologically active enzyme to produce a third product. This sequence of product/enzyme reactions may be repeated as many times as desired to achieve a given final product. This enzymatic circuit (factory) is only limited by the desired number of biosensor pairs and corresponding recognition sites on the DNA.

The above discussed and many other features and attendant advantages of the present invention will become better understood by reference to the following detailed description, when taken in conjunction with the accompanying drawings.

### BRIEF DESCRIPTION OF THE DRAWINGS

FIG. 1 is a diagrammatic representation of the basic method of the present invention showing the interaction of the two split enzymes (each with a mutually exclusive recognition molecule) with a target analyte to form a single enzyme biosensor.

FIG. 2 is a diagrammatic representation of an exemplary method of the present invention showing enhanced gain. The product of the interaction between Reactant #1 and the enzyme biosensor is an allosteric activator of an amplifier enzyme (#2). The allosteric activator activates the amplifier enzyme (#2), which in turn interacts with a second reactant (Reactant #2) to form an amplified product.

FIG. 3 is a diagrammatic representation of an exemplary embodiment of the present invention where a single biosensor is used to detect two different target analytes to provide a logic OR gate.

FIG. 4 is a diagrammatic representation of an exemplary embodiment of the present invention where three different biosensors are used in conjunction with two target analytes and three products to provide a logic AND gate.

FIG. 5 is a diagrammatic representation of an exemplary embodiment of the present invention wherein the split enzyme biosensor is used in an ELISA assay.

FIG. 6 is a diagrammatic representation of an exemplary embodiment of the present invention wherein the enzyme biosensor is used as in quantitative PCR.

FIG. 7 is a diagrammatic representation of exemplary detection systems in which may be used in conjunction with the enzyme biosensors in accordance with the present invention.

FIG. 8 is a diagrammatic representation of an exemplary live cell sensor assay in accordance with the present invention

FIG. 9 is a diagrammatic representation of the use of the present invention in an exemplary molecular evolution method (Parts I and II).

FIG. 10 is a continuation of the diagrammatic representation of the exemplary molecular evolution method set forth in FIG. 9 (Part I) and an alternate method.

FIG. 11 is a diagrammatic representation of the use of the biosensors of the present invention in an exemplary fluorescence in situ hybridization (FISH) assay.

FIG. 12 is a diagrammatic representation of the use of the biosensors of the present invention in an exemplary single nucleotide polymorphism (SNP) assay.

FIG. 13 is a diagrammatic representation of the use of the biosensors of the present invention in an exemplary single molecule DNA sequencing method.

FIG. 14 is a diagrammatic representation of the use of the biosensors of the present invention in an exemplary method for detecting protein-protein interactions and drug discovery.

FIG. 15 is a diagrammatic representation of the use of the biosensors of the present invention in an exemplary method for producing multiple products.

FIG. 16 is a diagrammatic representation of exemplary ScFv/Renilla constructs that may be used as the enzyme portion of the biosensors in accordance with the present invention.

FIG. 17 is a diagrammatic representation of exemplary ScFv/B-lactamase constructs that may be used as the enzyme portion of the biosensors in accordance with the present invention.

FIG. 18 is a diagrammatic representation of exemplary ScFv/β-galactosidase constructs that may be used as the enzyme portion of the biosensors in accordance with the present invention.

### DETAILED DESCRIPTION OF THE INVENTION

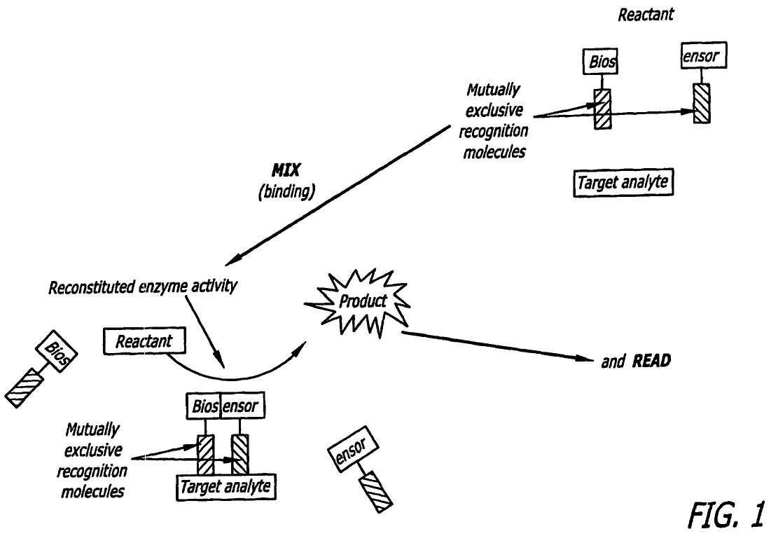

As set forth in FIG. 1, the present invention provides a split biosensor-linked immuno/nucleic acids detector which is solution-based, homogenous, “mix and read” that relies on dual-recognition (coincidence detection) of the analyte molecule coupled with enzymatic gain. This concept is very general and can be used to detect proteins, DNA, RNA, and many other kinds of macromolecules. The biosensor is composed of a split enzyme, where the two halves are linked to two recognition molecules, such as two single chain antibody fragments (ScFv), full antibodies, recognition peptides, DNA/PNA oligomers, aptamers, and the like. These recognition molecules bind to two, non-overlapping (‘mutually exclusive’ or “distinct”) epitopes of the same target analyte. Alternatively, they are linked to two ssDNA/PNA/aptamer oligonucleotides that are complementary and hybridize to a nucleic acid target sequence in tandem. Each ScFv/DNA/PNA/aptamer is linked through a non-degradable linker to a reconstitutible biosensor (split enzyme). Specifically, the biosensor is a molecule that has been split into two inactive portions. One portion of the biosensor is fused/covalently attached, through a linker, to one scFv/ssDNA/PNA/aptamer. The second portion of the biosensor molecule is fused/covalently attached, through a linker to the second scFv/ssDNA/PNA/aptamer.

The recognition molecules are designed to have very high affinity to their target. When the two recognition molecules bind to their target, the two portions of the biosensor molecule are brought into close proximity, establishing a reconstituted, measurable enzyme activity of the biosensor. This activity is based on turnover of a substrate molecule to a product molecule that gives rise to a new signal (fluorescence, bioluminescence, chemiluminescence, color change, de-quenching, pH, charge, electrical signal and the like), thus signaling the presence of the analyte/nucleic acid target. Depending on the concentration of reagents, incubation time and other reaction conditions, the signal can be made linear with the concentration of the target, providing very sensitive quantification of a target at extremely low dilution. This detection can be made in the milieu of strong background (serum, cell lysates, food, water, samples collected from air and the like) without any washing steps.

The detection can be performed on titrate plate readers, microscopes, fluorometers and many other commercially available readers. For some applications, new readers could be developed. Because of the simplified assay format, it should be possible to develop aerosol-based detection (first spray proteases to lyse cells, then spray reagents in accordance with the present invention as described herein) allowing to read out color change by the naked eye in-situ. This greatly simplifies analyte detection in the field. Inexpensive, consumer-friendly assays (analogous to existing home-pregnancy test kits as an example) may be prepared using the split enzyme biosensors of the present invention.

To prepare the biosensors in accordance with the present invention, enzymes of interest are cyclically split and screened for high reconstituted activity at high split concentration, but poor affinity for each other (low reconstituted activity) at low split concentration. The very high affinity of the two fused splits to their common target brings them in close proximity (increases the local concentration of the splits) and therefore provides high reconstituted activity. Only when both partners bind to the target, is there a measurable signal. This method requires coincidence (mutual binding) of the two halves to the same target, therefore enhancing reliability: it minimizes false negatives and false positives. It is a solution based, ‘mix and read’, homogenous assay. In many embodiments this assay does not require any washing steps, or subsequent enzymatic steps such as RT and PCR reactions. Using the biosensors in accordance with the present invention, it should be possible to detect a single mRNA or a single copy of a protein from a single cell.

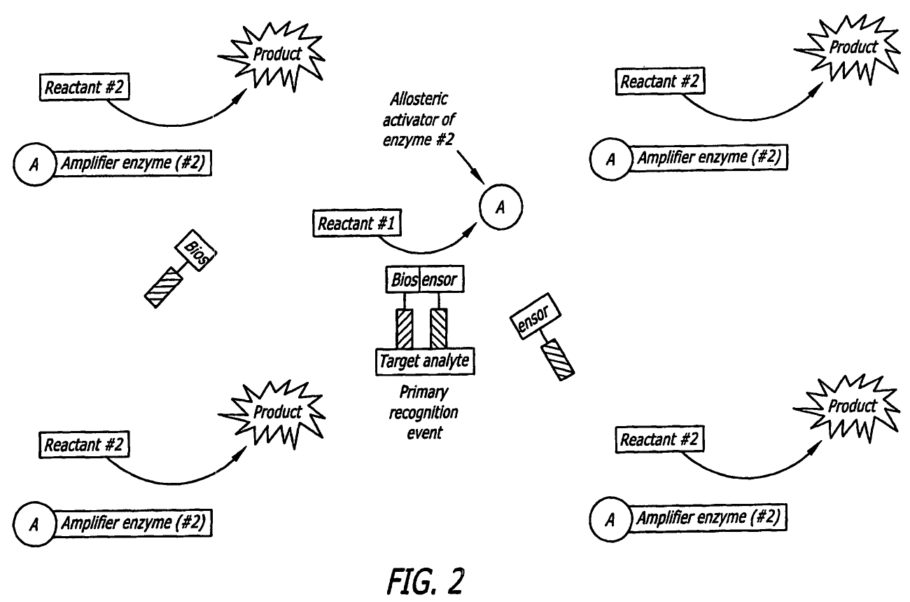

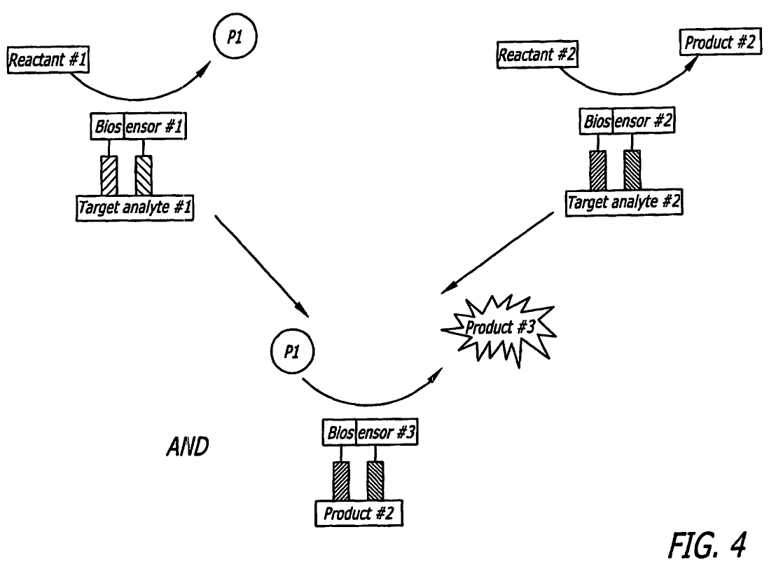

The method of the present invention mimics the cell's extreme sensitivity and specificity to activate a signaling cascade from a single molecular recognition event. As set forth in FIGS. 2 and 4, it combines the logical ‘AND’ gate that comes about from a dual molecular recognition (‘coincidence detection’), which provides enhanced specificity, together with enzymatic gain (multiple turn-overs). Cell signaling heavily relies on these two concepts. Often times, when ligands bind their receptors, they induce hetero-oligomerization (‘AND’ gate) and turn on enzymatic activity to produce many secondary messenger molecules (gain). Also, downstream in the signaling cascade, transcription factors frequently hetero-dimerize (‘AND’ gate) and bind to DNA to activate gene expression (possibly hetero-dimerization is mediated by the transcription factors monomers binding in tandem to a unique DNA sequence upstream from the promoter site). The activation process can be viewed as multiple turn-overs of RNAP clearing the promoter to synthesize mRNA.

As shown in FIG. 2, an amplifier enzyme (#2) may be used to provide enhanced gain, enhanced specificity, or both. For enhanced gain, the reconstituted split enzyme can turn over the substrate to a product (any suitable coenzyme) that allosterically controls another enzyme (free in solution, or possibly bound in close proximity to first enzyme). A single recognition event will generate many allosteric control molecules that will in turn activate many secondary enzymes that will now turn-over a very large number of ‘no signal’ substrate molecules to ‘yes signal’ product molecules. Such a scheme provides exponential gain at each amplification stage, and allows one to cascade several amplification stages (‘fan out’).

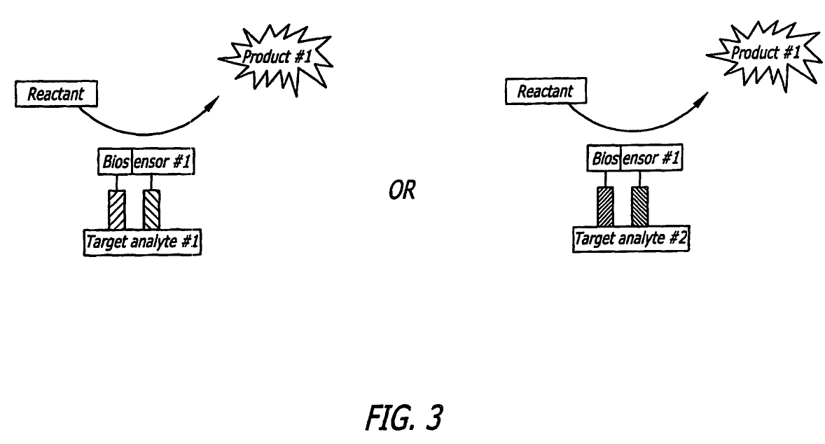

The present invention may be used to provide ‘signal processing’ capability like in electronics. Gain and ‘fan-out’ is described above. If one adds logical gates, there are enough components to build signaling circuits based on molecular recognition events. An OR gate (See FIG. 3) can be implemented by fusing two parts of the split enzyme to two scFv that recognize two orthogonal epitopes of analyte #1. The same two parts of the same split enzyme will also be fused to two scFv recognizing two orthogonal epitopes of analyte #2. Substrate (one kind) molecules are added to the solution. Detection of either analyte #1 OR analyte #2 will result in turn-over of substrate molecules into detectable product molecules.

An AND gate can be implemented by fusing two parts of the first split enzyme to two scFv recognizing two orthogonal epitopes of analyte #1. Two parts of a second (different) split enzyme is then fused to two scFv recognizing two orthogonal epitopes of analyte #2. Three different types of substrate molecules and a third kind of enzyme are then added to the solution. The third enzyme will depend on the two previous products to get activated and turn-over the third substrate into a detectable product, which indicates the presence of analyte #1 and analyte #2 in the solution. The two first products can be, for example, exposure of two epitopes for the split enzyme #3, and a substrate for it, or a substrate and allosteric binder that activates an unsplit enzyme #3. Several different permutations of these ideas can be used to construct the AND gate. As is apparent, sophisticated detection and signal processing of combinations of analytes can be achieved utilizing gain, fan-out, and logical gates as described above. In addition, these can be implemented inside (transfected) cells (substrate molecules have to be permeable), or outside, in solution.

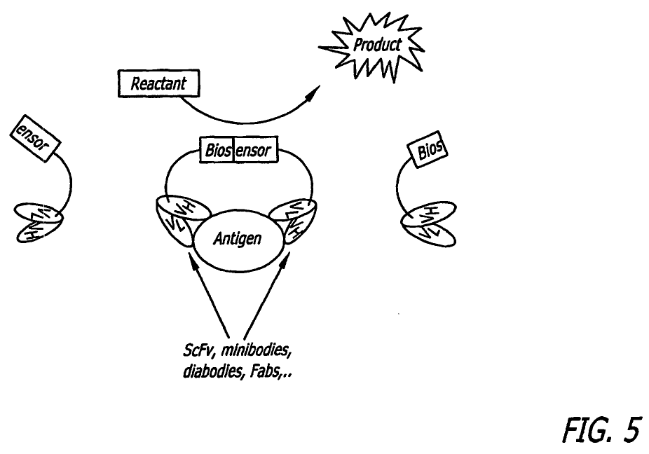

Referring to FIG. 5, antibodies are generated by B lymphocytes in the mammalian immune system, and are comprised of two light and two heavy polypeptide chains, that are disulfide linked in a “Y” structure. The antigen-combining site resides at the tips of the Y, and is formed by the heavy chain variable domain and light chain variable domain coming together. These variable domains are composed almost entirely of antiparallel beta sheet (Fab structure, Satow et al., 1986; Marquart et al., 1980). Hypervariable loops at ends of beta strands, also known as the complementarity-determining regions (CDRs), are responsible for the broad diversity of binding specificities exhibited by native antibodies.

Generation of recombinant antibodies through the co-expression of the entire heavy and light chains (Cabilly, et al., 1984; Boss, et al., 1984) resulted in high levels of protein production, but no binding of antigen until proteins were refolded. Better yields of refolding Fab fragments and better still for Fv fragments (Huston et al, 1988).

Many researchers have successfully designed and constructed single chain antibodies (scFv) composed of a variable heavy (V_{H}) recognition domain and a variable light domain (V_{L}) (Huston, Levinson et al. 1988). These domains, when linked with a short peptide linker of approximately 15-20 residues, have been shown to fold properly and to bind to their respective antigens without refolding. Most native murine antibodies can be successfully converted to scFv.

New technologies, such as phage/ribosome/yeast displays provide an alternate method for directly isolating scFvs with the desired binding specificity (Marks, Hoogenboom et al. 1991) (Hanes, Jermutus et al. 1998) Feldhaus, et al., 2003). Large libraries of bacteriophage, each “displaying” a unique, random scFv as a fusion protein on its surface, can be “panned” on target antigen or cells, and phage displaying scFvs which bind specifically to the target antigen or cell can be identified. If the starting library is large enough (10⁹-10¹³), antibodies with high affinity can be directly isolated (at least 10⁻⁹ M, similar to natural antibodies). Affinity maturation by yeast display can readily optimize these scFvs to sub-picomolar affinities (Feldhaus, et al., 2003). Antibodies can readily be derived with specificity toward non-conventional antigens that are not amenable to conventional murine hybridoma technology, including “self” antigens, toxic substances, small molecules, etc. Using such display technologies, multiple epitopes of the same target can be identified, mapped, and mutually exclusive (“orthogonal”) binders can be identified.

Thus, given a target antigen, it is straightforward to generate a set of non-overlapping scFv that bind with high affinity and specificity. Each of two scFv binding non-overlapping epitopes on the target antigen can then be fused to the two halves of a split enzyme using a linker peptide. Simultaneous binding of both scFv to the same antigen molecule will allow reconstitution of the split enzyme activity and generation of a signal.

Furthermore, the recognition molecules in the split detector can be constructed from the individual V_{H} or V_{L} domains from an antibody that binds to the desired target. These chains could be engineered to maintain low individual affinities to the target and to only achieve high affinity when both domains bind simultaneously to the target (reconstituting the Fv on the target). Fusion of the separate VL or VH domains to the halves of the split enzyme partner would ensure generation of signal only upon simultaneous binding. These detectors would have a definite advantage for small analytes (compared to scFvs that recognize larger epitopes).

Many native and modified native peptide sequences have been identified with high affinity, highly specific binding to their corresponding receptor proteins. These include peptide hormones and peptide neurotransmitters. An example is somatostatin and the somatostatin receptors, and synthetic somatostatin analogs that can be used as drugs. Furthermore, with the advent of phage display and other display technologies described above, it is straightforward to screen random libraries of peptide sequences and isolate novel peptides with high affinity to the antigen target of choice. Peptides can be either linear or constrained loops (made by including two cysteine residues at a fixed or variable distance apart in the peptide sequences). Furthermore, it is straightforward to identify sets of peptides that bind to the target antigen in a non-overlapping fashion. These peptides can then be fused to the corresponding split enzyme partner, using a linker peptide sequence as needed to provide the correct spacing and orientation. Simultaneous binding of the two peptide-enzyme fusion proteins will reconstitute the enzyme activity and generate signal. In summary, this approach generates and utilizes peptide probes that are suitable for sensing protein/antigen targets as shown in FIG. 5.

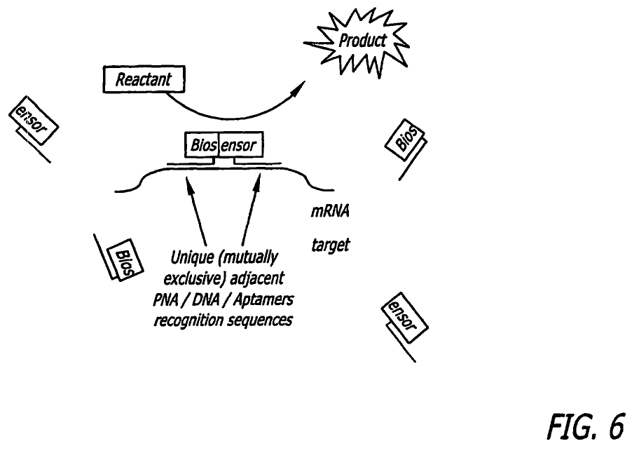

As shown in FIG. 6, oligonucleotide probes (short synthetic nucleic acids, typically 20-50 nucleotides in length) can be designed to be highly specific for target DNA or RNA sequences. Furthermore, one can design two adjacent oligonucleotide probes, which can then be linked to the two halves of a split enzyme. Simultaneous binding of both oligonucleotides to the same target DNA or RNA sequence will allow reconstitution of the split enzyme activity and generation of signal.

The oligonucleotide probe itself can have different chemical compositions. Native DNA oligonucleotides have phosphodiester bonds. Modifications can be introduced to improve stability or binding affinity. Modifications can include phosphorothiolate oligonucleotides, or modified RNA oligonucleotides such as 2′-O-methyl- or 2′-F-ribo-oligonucleotides, and oligonucleotides with modified bases. These are readily produced using DNA synthesizer instruments. Another class of related probes are peptide nucleic acids (PNA). These are analogous to nucleic acids except the backbone consists of polypeptide linkages rather than sugar-phosphate linkages. Attached to the peptide backbone are standard, or modified, nucleoside bases. PNA probes are readily produced using peptide synthesizer instruments.

In order to attach the subunits of the split enzymes to DNA/PNA probes, conjugation methods can be used. In particular, modified bases are incorporated that provide a unique chemically reactive group (such as a thiol or amino group) at one end of the oligonucleotide. Furthermore, the two halves of the split enzyme can similarly be engineered to contain unique cys, lys, intein ligation chemistry, or other chemically reactive residues to allow reproducible modification at a specific site on the protein. This will allow for production of homogenous oligo-enzyme conjugates with defined stoichiometry and orientation. Standard protein chemistry approaches (using bifunctional linkers) are used to conjugate each oligonucleotide with its corresponding split enzyme fragment to generate the desired reagents. In summary, this embodiment of the invention generates and utilizes nucleic acid-based probes specific for nucleic acid targets.

Aptamers are defined nucleic acid sequences, longer than oligonucleotides, that fold into defined three-dimensional structures. Typically aptamers are 20 to over 100 nucleotides in length, and fall between peptides and proteins in terms of molecular weight. Using a process called SELEX random libraries of aptamers can be generated and selected for specific, high affinity binding to the target protein of interest. Thus, aptamers are large, defined, structured nucleic acid molecules with binding properties analogous to antibodies. As shown in FIG. 5, the present invention can be used to generate a set of aptamers that bind independently to the target, in a non-overlapping fashion. Pairs of aptamers can then be conjugated to the two halves of the split enzyme as described in the previous section on oligonucleotide probes.

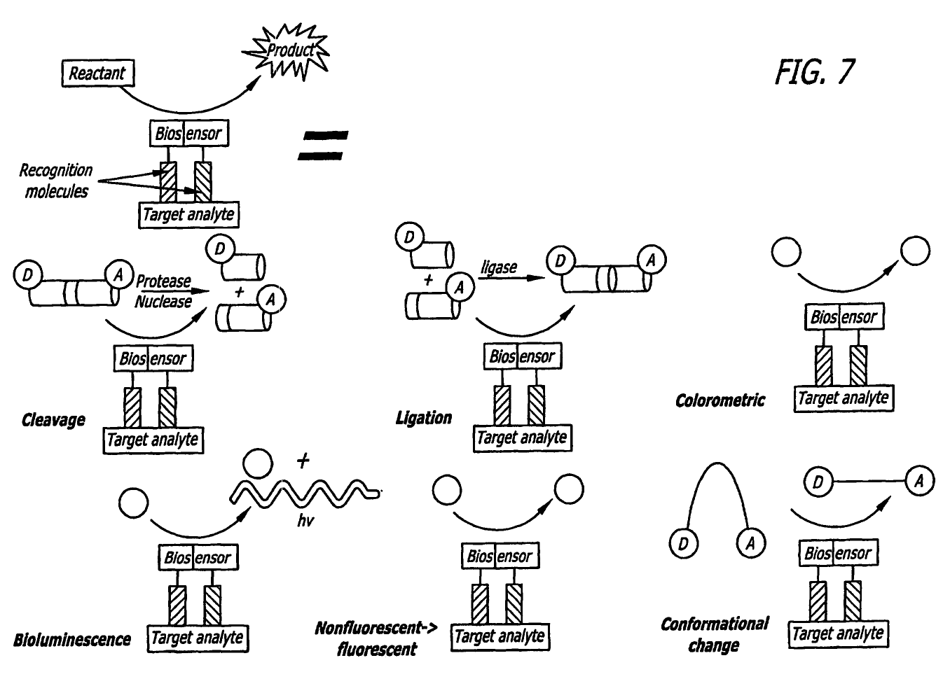

The biosensors in accordance with the present invention include split enzyme molecules that have a low affinity for one another, but will complement each other when brought into close spatial proximity (local high concentration). These biosensors are enzyme molecules that turn over “no signal” substrate molecules to “yes signal” product molecules. As shown in FIG. 7, the product molecules can be easily monitored by fluorescence, bioluminescence, chemiluminescence, colorometric, de-quenching, pH, charge, electrical signals, and others. It is important that the splits have low afity for one another to increase the signal-to-noise ration, thus eliminating unwanted background. Attenuation of affinity can be accomplished by engineering residues at the interacting surfaces of the splits.

The biosensors of the present invention include, but are not limited to, enzymes that can be split into two independent folding domains, which by themselves have little or no detectable activity, nor appreciable affinity for their complementary partner. However, once brought together by the affinity of the detector for its analyte, activity is restored. Because of the enzymatic turn-over of a measurable product, this assay provides gain (amplification) to the signal associated with the presence of the targeted analyte.

There is a growing list of split enzymes that can be utilized for this function. The work of Paulmurugan and Gambhir (2003) describe a split Renilla luciferase that can be reconstituted and monitored via bioluminescence. Also see U.S. Pat No. 5,292,658. The work of Rossi, et al. (1997) describe the nonfunctional, complementing split B-galactosidase. Complementing B-galactosidease activity can be monitored via colorometric, chemiluminescence, or fluorescence detection. Split B-lactamase has been described by Spotts, et al. (2002) whose complementation was assayed by the color change of nitrocefin upon hydrolysis or by fluorescence via CCF-2/AM (Vertex).

Studies of other enzymes such as GTPases (change of charge), peroxidases (colorometric), nucleases (endo and exo cleavage), restriction endonucleases (sequence specific endo cleavage), proteases (protein cleavage), ligases (ligating nucleic acid oligos), thiol-disulfide oxidoreductases (conformational change through disulfide bonds) and many more are suitable for use as split enzymes, and corresponding substrate molecules, using similar methods, to generate “no signal” to “yes signal” turn-over with amplification, that can be easily monitored by the detection methods described in FIG. 7. In addition, protein interfaces of the splits can be engineered to modulate their interactions.

The list of possible substrates, fluorophores, and the like, is also rapidly growing. Korlach et al. (2004) describe the development of guanosine 5′-triphosphate analogs that allow the fluorescent-based monitoring of GTpase activity. Galactosidase activity can be measured by colorometic assays with BCIP/NBT or ONPG, chemiluminescence using the Galacto-Light Plus assay kit (Tropix) or fluorescence via Fluor-X-Gal or fluorescein di-B-D-galactopyranoside. Other substrates include Luciferin for luciferases, 10-acetyl-3,7-dihydroxyphenoxazine, and fluorophores that change spectrum/quantum yield upon binding to the sensor (as, for example, antibodies against fluorophores that quench them upon binding). In addition, molecular beacons may be used to provide a valuable substrate for a split restriction endonuclease. The molecular beacon may contain two strands of complementary DNA, or a DNA hairpin. A detector moiety, such as a fluorophore is attached to one end of one oligonucleotide, while a quencher moiety is attached to the complementary strand. When double-stranded, the fluorescence is quenched. However, upon reconstituting the restriction enzyme via binding to the analyte, the DNA is cut, lowering the Tm, resulting in DNA denaturation, and fluorescence emitted. Molecular beacons have been described (Yao, et al., 2003; Yates, et al., 2001).

Further, the above assays can be multiplexed with several biosensors and different substrates to test for various combinations or nuances of target analytes. For example, libraries of split enzymes that can turn over corresponding substrates to different color products can be made. The basic assay could then be multiplexed: the presence of a particular color fingerprint at the end of the assay will indicate the presence of a subset of analyte molecules from the tested pool of molecules.

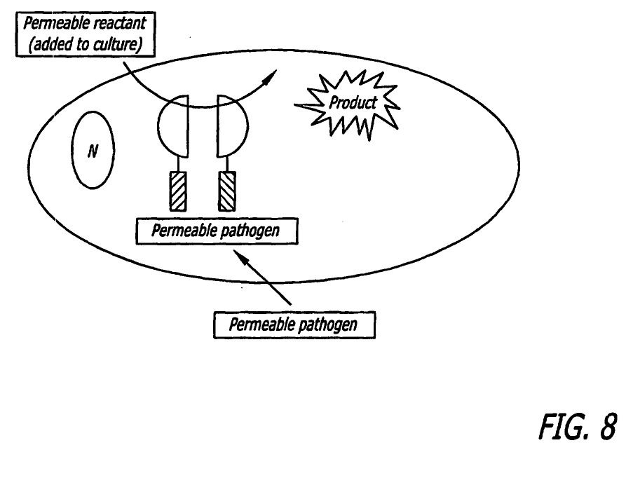

As shown in FIG. 8, the method of the present invention can be implemented in live cells, in tissue culture, and possibly even in the living body, using transient transfection, stable transfection or gene delivery/gene therapy methods. In this embodiment, biosensors with scFv (C.1.) or peptides (C.2) recognition parts are genetically introduced into the cell and endogenously produced by the cell itself. If the recognition parts are nucleic acid based (C.3. or C.4), conjugated to the split enzyme parts), the biosensor parts have to be brought from outside the cell and cross the plasma membrane by conventional protein transfection methods. The two halves are silent (no signal). Only when the cell synthesizes the target (mRNA/protein), signal will be generated. This way mRNA and protein levels could be monitored and quantified in real time, down to the single cell, single copy level. Possibly, with 2 enzymes, 2 colors, a ratio of protein to mRNA levels could be monitored in real time. Cell permeable substrate molecules will be used in this case (added to the culture medium or to the blood (for in vivo applications). Utilizing gene therapy methods, particular cells in the body could be targeted (for example, cancerous cells) and unique marker/target level could be monitored. Here in addition to all other substrate molecules, radiolabels could be turned-over to stay in the target cell and provide molecular imaging contrast.

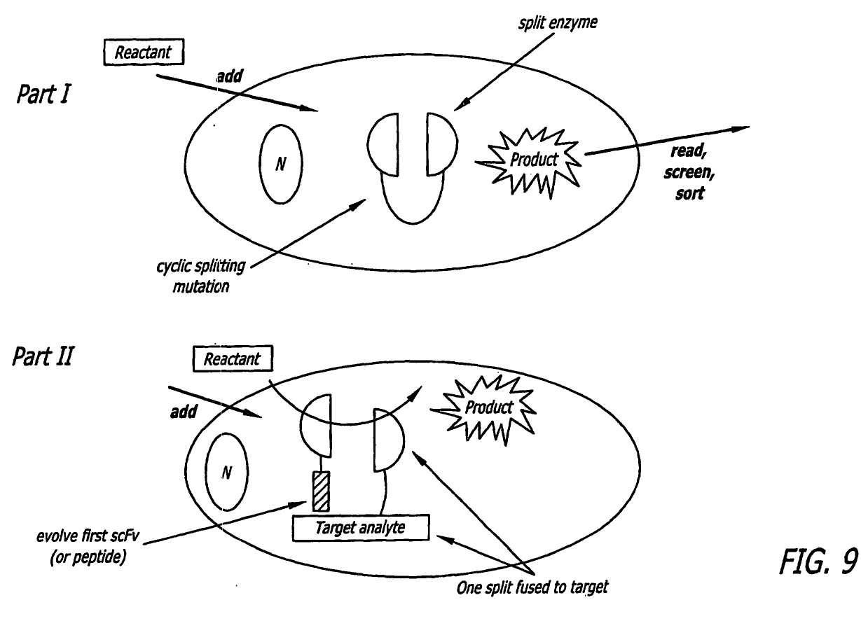

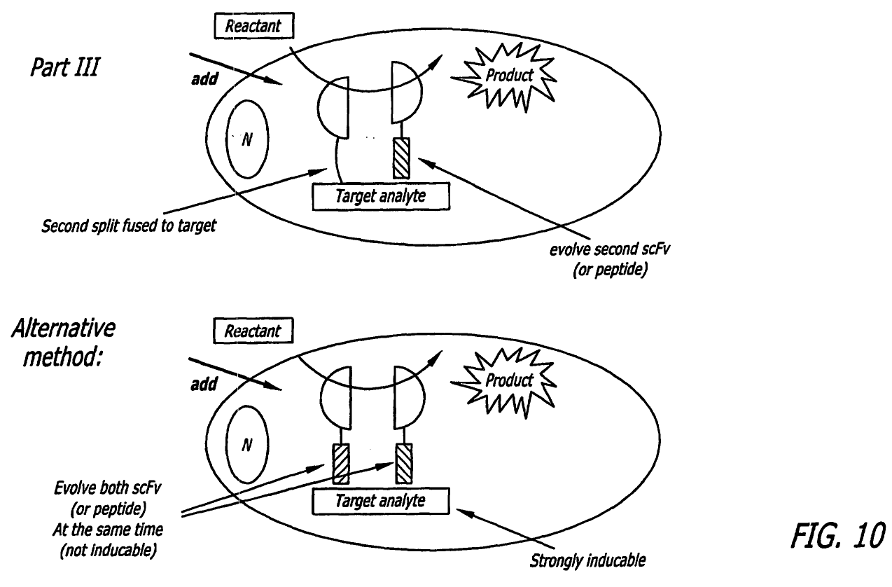

As shown in FIGS. 9 and 10, the present invention may be used for carrying out molecular evolution. All reagents can be evolved and affinity maturated in live cells and tested for desired action in the cell (provided that the substrates are cell permeable). The two halves of the biosensor could be endogenously expressed (non-inducible promoter). The target analyte, will be strongly induced. The split enzyme parts will be fixed, but scFv or peptide recognition parts will be randomly mutated to generate a large library. After adding permeable substrates, cells will be screened and sorted based on two selection pressures: no signal with no analyte induction, strong signal with analyte induction. Possible strategies and the steps for the evolution are as follows:

1. evolution of split enzymes

2. evolution of first antibody

3. evolution of second antibody

4. evolution of linker.

Some steps can be shortcut. For example, after steps #1 and #2, one could express simultaneously all parts: antigen and two split sensors. Evolve a second antibody and look for the turn-over signal. Cells expressing two soluble splits, but no antigen, can be smart detectors for permeable analytes.

As an alternative embodiment, the assay will be developed for secreted proteins (secretion signals added to the two splits), and analytes need not be permeable. In this case, cells need to be adherent and in a small well format. The winners will be pre-sorted by adding another expression indicating successful assembly (for example, avidine expression on the cell wall combined with magnetic beads presorting) and then sorted for example, by optical signal, on a FACS (assuming fluorescence signal) or a microscope, and affinity maturation will be achieved after several generations. This type of display could generate many split enzyme sensor pairs for numerous different targets, for several mutations of the second split (while keeping the first split constant), and for different enzymes, generating a large toolbox suitable for multiplexed detection. The splits themselves, for various enzymes, could be evolved in a cyclic mutation, looking and sorting for strongest signal. This display could be performed in the periplasma of e-coli and/or in yeast. Positive results will allow one to move to production of reagents (by cloning and purification) for in-vitro uses. The same cell lines can be used as smart detectors (simpler liquid handling for fieldable detectors).

This invention introduces many advantages to bio-detection: Sensitivity, specificity, simplicity, use of simple (existing) readers, homogenous, “mix and read” format. The solution-based, dual-recognition, split-enzyme linked immuno/nucleic acid detector system will have broad applications where highly specific, sensitive, inexpensive and portable detection of specific biological agents or substances are required. For example, in the field, these biosensors could be used to detect agents of bio-terrorism, such as noxious bacteria (either presence of their DNA, or bacteria-specific proteins) and their toxins (anthrax, botulism toxin, ricin toxin). Biosensors in accordance with the present invention can also be made for rapid, sensitive detection of virulent, pathogenic viruses such as smallpox, Ebola and the like. (based on the detection of viral DNA/RNA or virus-specific proteins). These assays could be performed in solution, after specimen collection and cell lysing. Furthermore, since the assay is solution-based, the reagents could be sprayed onto surfaces where suspected bacteria/toxins/viruses were located. The evolution of color/fluorescence/bioluminescence would indicate the presence of the agent in question.

With respect to environmental situations, biosensors can be made to identify toxins and contaminants in water, air and soil. There are also potential applications in food/agriculture, such as detection of E. coli 01H7 in meat or in plant crops and the potential of developing sensors for agents that cause diseases such as mad cow disease.

In the clinical setting, applications for the present invention are numerous. These include broad utility in infectious disease, where biosensors could be used to identify the presence and identity of viral or bacterial infections, including sexually transmitted diseases (STDs). Furthermore, clinical applications extend to chronic diseases, such as diabetes, heart disease, and autoimmune conditions. For example, in cardiovascular disease, currently the presence of serum markers such as C-reactive peptide or LPLipase A have been identified as predictive of high risk for heart attack and additional markers are being identified through genomics/proteomics research. The split enzyme biosensors described herein would provide rapid, sensitive detection of these markers.

Biosensors in accordance with the present invention also find utility in acute settings, such as rapid determination as to whether heart attack or stroke has occurred. In oncology, there are many examples of tumor-associated antigens or tissue-specific antigens that are useful markers/targets in cancer: breast (Fendly et al., 1990), lung (Hellstrom et al., 1990), colon (Yokota et al., 1992). Again, new information from genomics/proteomics research is rapidly adding to the list of markers that would be useful to identify using the split enzyme biosensors of the present invention. Biosensor-based detection can be envisioned to play an important role in screening/early detection of cancer, detection of recurrence, planning targeted therapy, and monitoring response to therapy. In reproductive biology, biosensor devices would provide a fast and facile method for evaluation of hormone levels, to better pinpoint ovulation, fertilization, and other key events.

In the arenas of diagnostics and drug discovery, biosensors in accordance with the present invention can play an important role both in research, and ultimately in final implementation. Initially the biosensor molecules described herein can be used for expression level analysis at the mRnNA and protein level, and the quantification of mRNA message and its corresponding translated protein both at the same time, on same cell lysate. Once the utility of target measurement is validated, practical devices based on these biosensor molecules can be manufactured and potential provide an inexpensive, rapid, and sensitive means for assessing specific mRNAs and proteins.

In PCT Application No. US04/19709, the coincident detection of single molecules with no enzymatic gain is demonstrated. The key requirement for such measurements is a small detection volume, as afforded for example, by confocal microscopy or total internal reflection (TIR) microscopy. Fluorescence correlation spectroscopy and/or burst detection techniques afford the detection of femtoMolar (fM) concentrations of analytes in femtoLiter (fL) volumes. The enzymatic gain (one amplification stage or more) could afford the detection of much lower concentrations of analytes in the same volume, or similar concentrations in much larger volumes (simplifying the instrumentation needed for detection by alleviating the need for sophisticated confocal/TIR optics). This improved sensitivity will allow the detection of individual macromolecules from a single lysed cell. Liquid droplets as small as 100 nL can be manipulated and handled by electrowetting-on-dielectric. Dilution and mixing steps could be performed on such small droplet and the amplified result could be read from a single cell, single molecule experiment.

The biosensors of the present invention can be used for detailed and very sensitive analysis of plasma proteins for diagnostics and pharmacogenomic applications. Bacterial and viral infections, sepsis and the like, could be tested by drawing blood from patients at the bedside and getting the results within a few minutes. DNA/RNA and proteins from the same pathogens could be tested simultaneously, increasing the reliability of the test.

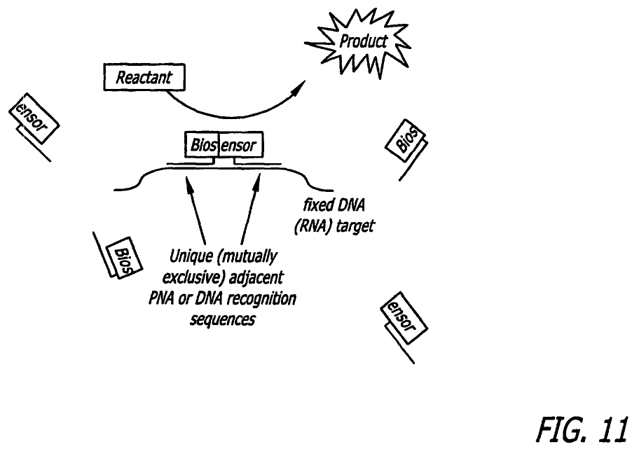

Genetic disorders can be monitored by detecting two adjacent sequences using the split enzyme sensors, as described above. Conventional fluorescence in situ hybridization (FISH) requires fixation of chromosome preparations. Utilizing the sensors of the present invention, FISH could be performed in solution (See FIG. 11). If a fixed form is needed, product molecules can be made to stick to surface after turn-over or reconstituted enzyme can polymerize a long tethered chain of product molecules that are directly detected or can be “developed” in a single, simple step.

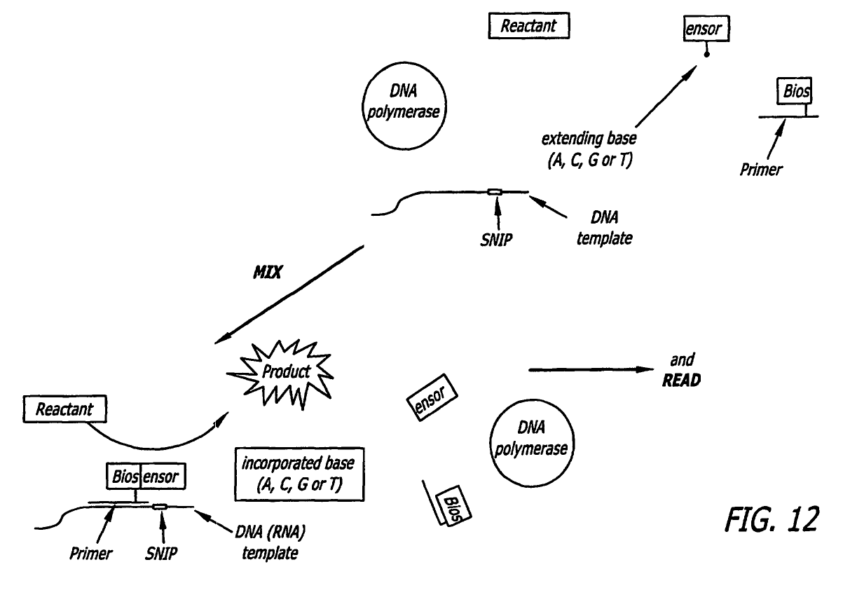

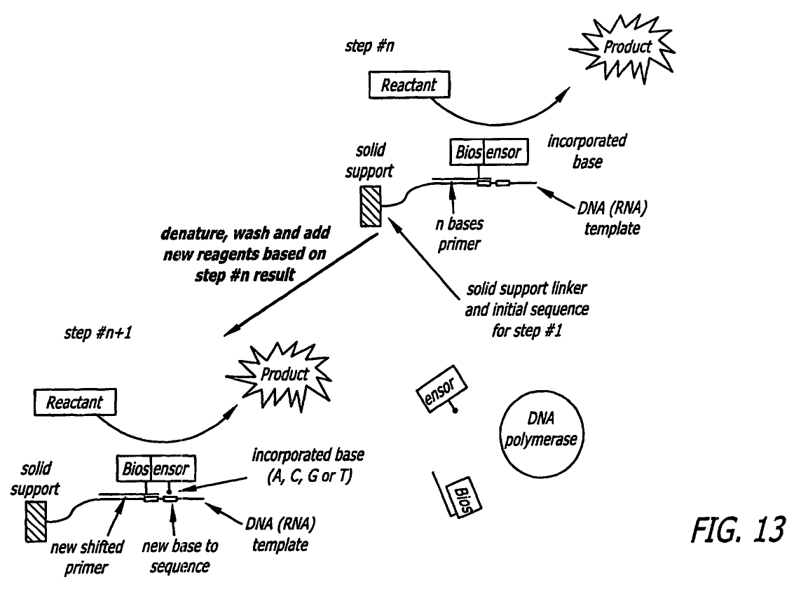

Referring to FIGS. 12 and 13, a genomic DNA can be prepared by ligating to the end a spacer with a given sequence and then tethering it to a solid support. The first split enzyme will target the ligated sequence. The second split enzyme will have a tethered single base (4 different reagents representing the 4 bases) as the recognition molecule. DNA polymerase will be added. This process will be repeated up to 4 times until successful incorporation of the base, constituting the reading of the first base in the unknown sequence. The biosensor will be removed by denaturation and a washing step. Based on the result of the first step, a new split biosensor with the appropriate conjugated (same length) primer will be added. The process will be repeated for the second position (with 4 reagents), incorporation, denaturation and washing. This process will be repeated until the whole sequence is read. This process requires a library of split-primer conjugates with 4ⁿ different primers, where n is the length (number of bases) of the primer. The challenge is in fluid manipulation (many steps) and the size of the library, which depends on n. Steps can be saved by having 4 different second splits that are complementary to the same first (primer) split that give rise to 4 different signals. Also, the extension step can be 2 or 3 bases at a time.

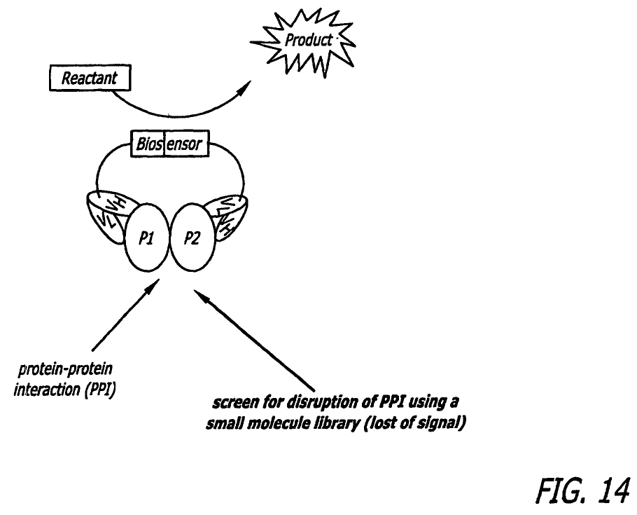

As shown in FIG. 14, the present invention may be used to screen for protein-protein interactions (PPI) and for their disruption. All soluble proteins in an organism are expressed and purified. An ScFv against each protein is developed. Two libraries are constructed: all proteins bound to their scFv with first split fusion (the ‘bios’ part of the sensor) and all proteins bound to their scFV with second split fusion (the 'ensor part of the biosensor). Then one member at a time of first library is screened against all members of second library, until all possible pair-wise interactions are screened. For positive interactions, it is then possible to assay for disruption (evidenced by loss of a positive detection signal) with a small molecule (drug candidate) library.

For in-vivo use, when the two split enzymes (‘bios’ and ‘ensor’) find a target, they reconstitute activity of a therapeutic enzyme to provide targeted enzyme therapy. Enzymes which may be targeted in this way include proteases. The invention will also work for dual detection of the soluble part of a cell marker in bodily fluids (displaying two orthogonal epitopes) or two close-by markers in the outside of the cell membrane. Alternatively, the method can be used for gene therapy/gene delivery techniques to introduce the DNA of the a split enzyme into cells in humans; the DNA will be transcribed and the mRNA will be translated; the split enzyme will be activated only if the antigen is detected inside the cell (for example, protease that will trigger apoptosis based on detection of a tumor marker). This same kind of approach could be used to convert non-self to self and self to non-self in the treatment of auto-autoimmune diseases.

The above concept of dual (coincidence) detection for activation of a drug will increase fidelity of targeted delivery of therapeutics. A sophisticated drug activation cascade could be implemented in the body using the logic gates and gain as also described above. For example, a logic statement for drug (protease) activation could be: activate only if analyte #1 and analyte #2 are present, but analyte #3 is not present, where the analytes are known markers for diseases.

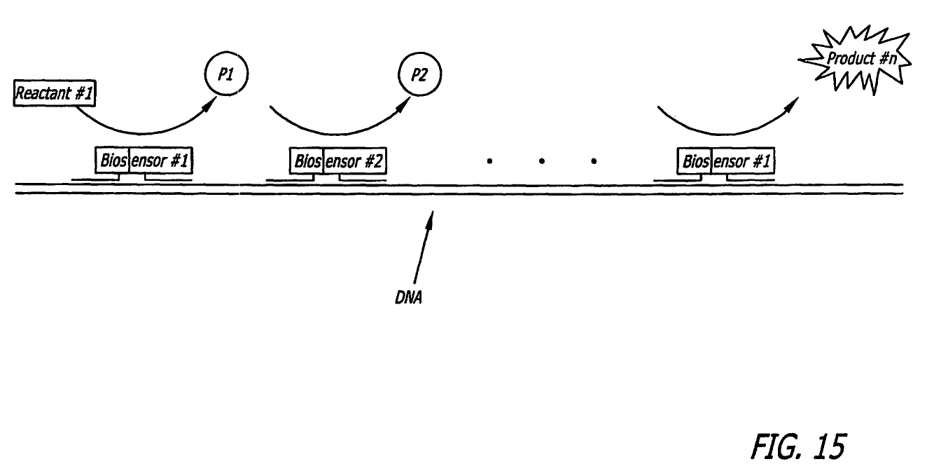

Referring to FIG. 15, a whole enzymatic cascade (as in metabolic circuits in the cell) could be constructed on DNA (immobilized on surface or in solution). Split enzyme biosensors recognizing adjacent sequences could be located one next to each other by hybridization. The coincidence requirement of the split for reconstitution of enzyme activity will increase reliability (minimizing non-specific binding). Substrate (Reactant #1) for a first enzyme (Biosensor #1) in the cascade is added. This produces (turns-over into) a product (P1) that is a substrate for second enzyme (Biosenor #2) which produces a second product (P2). The process is repeated along the DNA chain for n Biosensors (Biosensor #n) to produce n products (Product #n). Gain could be introduced in between steps (to compensate for products diffusing away), as described above. If DNA is combined in a linear fashion on the support, the reaction cascade can be controlled spatially so that the substrate is provided at one end and the final product produced at the other end. This method could be combined with microfluidics for liquid manipulation.

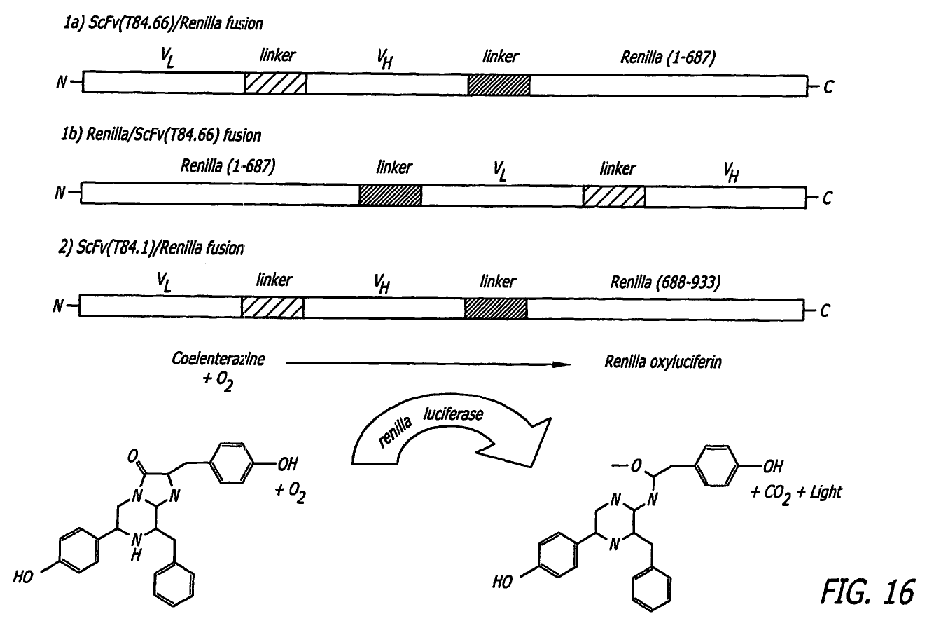

Several exemplary constructs, as shown in FIGS. 16-18, can be generated for expression (either intracellular or secreted) in E. coli and use as the split enzyme portion of the biosensor. Included are hexahistidine-tagged chimeras of split enzymes fused to anti-CEA scFvs (Wu, Chen et al. 1996). The parental anti-CEA antibodies are described in (Hammarstrom, Shively et al. 1989) and references therein. These constructs are diagramed in FIGS. 16, 17 and 18.

The first construct (FIG. 16-1a and 1b) contains anti-CEA scFv (T84.66) fused either at the N-terminus (1a) or C-terminus (1b) to split renilla luciferase (residues 1 to 687). The partner chimera (FIG. 16-2) is the construct containing anti-CEA scFv (T84.1) fused at the C-terminus to split renilla luciferase (residues 688 to 933). It is known from previous studies (Paulmurugan and Gambhir, 2003) that these Renilla constructs can be reconstituted and can be monitored via bioluminescence. Similarly, bioluminescence will be measured following binding of the scFvs to CEA.

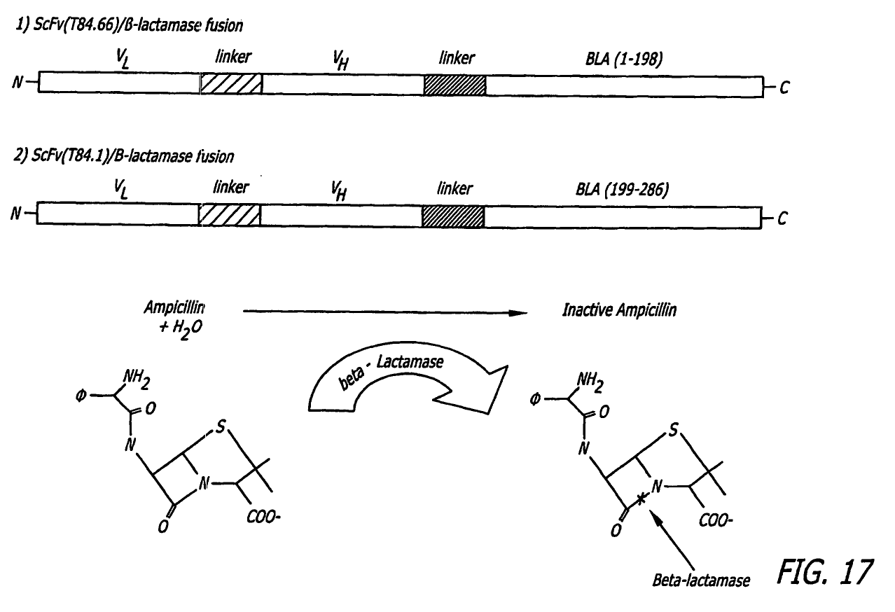

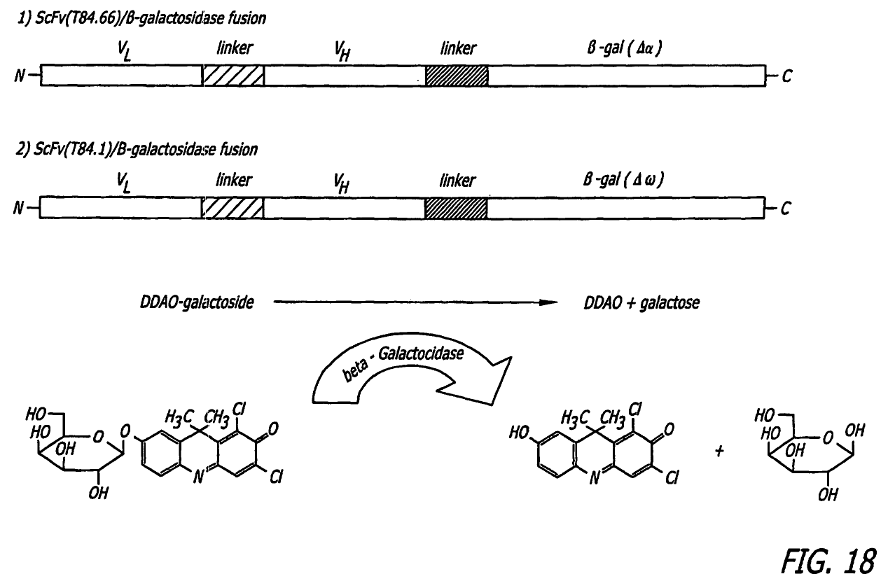

Similar constructs, which include anti-CEA scFv (T84.66) and anti-CEA scFv (T84.1) will be made using split enzymes of B-lactamase and B-galactosidase. See FIGS. 17 and 18, respectively. A recombinant form of the CEA antigen called CEA N-A3 has been generated, comprising both binding sites (You, Hefta et al. 1998). T84.1 binds to the N-domain and T84.66 binds to the A3 domain. This antigen can be used as model target analyte to test the split enzymes for formation into a suitable biosensor when they interact with the CEA N-A3.

Although the foregoing invention has been described in some detail for purposes of clarity of understanding, those skilled in the art will appreciate that various adaptations and modifications of the just described preferred embodiments can be configured without departing from the scope and spirit of the invention. The described embodiments should be taken as illustrative and not restrictive, and the invention should not be limited to the details given herein but should be defined by the following claims and their full scope of equivalents.

### BIBLIOGRAPHY

Boss, M. A., Kenten, J. H., Wood, C. R., and Emtage, J. S., Assembly of functional antibodies from immunoglobulin heavy and light chains synthesized in E. coli. Nucleic Acids Res. 1984 May 11; 12(9):3791-806.
Cabilly, S., Riggs, A. D., Pande, H., Shively, J. E., Holmes, W. E., Rey, M., Perry, L. J., Wetzel, R., and Heyneker, H. L., Generation of antibody activity from immunoglobulin polypeptide chains produced in Escherichia coli, Proc. Natl. Acad. Sci., USA, 81:3273-3277, 1984.
Feldhaus, M. J., Siegel, R. W., Opresko, L. K., Coleman, J. R., Feldhaus, J. M., Yeung, Y. A., Cochran, J. R., Heinzelman, P., Colby, D., Swers, J., Graff, C., Wiley, H. S., Wittrup, K. D., Flow-cytometric isolation of human antibodies from a nonimmune Saccharomyces cerevisiae surface display library., Nat Biotechnol. 21:163-70, 2003
Fendly, B. M., Kotts, C., Vetterlein, D., Lewis, G. D., Winger, M., Carver, M. E., Watson, S. R., Sarup, J., Saks, S., Ullrich, A., et al., The extracellular domain of HER2/neu is a potential immunogen for active specific immunotherapy of breast cancer, J Biol Response Mod. 9:449-55, 1990
Hammarstrom, S., J. E. Shively, et al. (1989). “Antigenic sites in carcinoembryonic antigen.” Canc. Res. 49: 4852-4858.
Hanes, J., L. Jermutus, et al. (1998). “Ribosome display efficiently selects and evolves high-affinity antibodies in vitro from immune libraries.” Proc. Natl. Acad. Sci. USA 95: 14130-14135.
Hellstrom, I., Garrigues, H. J., Garrigues, U., Hellstrom, K. E., Highly tumor-reactive, internalizing, mouse monoclonal antibodies to Le(y)-related cell surface antigens, Cancer Res. 50:2183-90, 1990.
Huston, J. S., D. Levinson, et al. (1988). “Protein engineering of antibody binding sites: Recovery of specific activity in an anti-digoxin single-chain Fv analogue produced in Escherichia coli.” Proc. Natl. Acad. Sci. USA 85: 5879-5883.
Huston, J. S., Levinson, D., Mudgett-Hunter, M., Tai, M. S., Novotny, J., Margolies, M. N., Ridge, R. J., Bruccoleri, R. E., Haber, E., Crea, R, and Oppermann, H. Protein engineering of antibody binding sites: recovery of specific activity in an anti-digoxin single chain Fv analogue produced in Escherichia coli, Proc. Natl. Acad. Sci. USA, 85:5879-5883, 1988.
Korlach, J., Baird, D. W., Heikal, A. A., Gee, K. R., Hoffman, G. R., and Webb, W. W., Spontaneous nucleotide exchange in low molecular weight GTPases by fluorescently labeled {gamma}-phosphate-linked GTP analogs, Proc. Natl. Acad. Sci. USA, 2004.
Marks, J. D., H. R. Hoogenboom, et al. (1991). “By-passing immunization: Human antibodies from V-gene libraries displayed on phage.” J. Mol. Biol. 222: 581-597.
Marquart, M., Deisenhofer, J., Huber, R., Palm, W., Crystallographic refinement and atomic models of the intact immunoglobulin molecule Kol and its antigen-binding fragment at 3.0 A and 1.0 A resolution, J Mol Biol. 141:369-91, 1980
Paulmurugan, R. and Gambhir, S. S., Monitoring protein-protein interactions using split synthetic renilla luciferase protein-fragment-assisted complementation, Analytical Chemistry, 75: 1584-1589, 2003.
Rossi, F., Charlton, C. A., Blau, H. M., Monitoring protein-protein interactions in intaqct eukaryotic cells by B-galactosidase complementation, Proc. Natl. Acad. Sci., 94: 8405-10, 1997.
Satow, Y., Cohen, G. H., Padlan, E. A., Davies, D. R., Phosphocholine binding immunoglobulin Fab McPC603. An X-ray diffraction study at 2.7 A, J Mol Biol. 190:593-604, 1986
Spotts, J. M., Dolnetsch, R. E., Greenberg, M. E., Time-lapse imaging of a dynamic phosphorylation-dependent protein-protein interaction in mammalian cells, Proc. Natl., Acad. Sci., 99: 15142-47, 2002.
Wu, A. M., Chen, W., Raubitschek, A., Williams, L. E., Neumaier, M., Fisher, R., Hu, S-z., Odom-Maryon, T., Wong, J. Y. C., and Shively, J. E. Tumor localization of anti-CEA single-chain Fvs: improved targeting by non-covalent dimmers, Immunotechnology, 2: 21-36, 1996.
Yates, S., Penning, M., Goudsmit, J., Frantzen, I., Weijer, B., Strijp, D., Gemen, B., Quantitative detection of Hepatitis B virus DNA by real-time nucleic acid sequence-based amplification with molecular beacon detection, J. Clin. Micro., 39: 3656-65, 2001.
Yao, G., Fang, X., Yokota, H., Yanagida, T., Tan, W., Monitoring molecular beacon DNA probe hybridization at the single molecule level, Chem. Eur. J., 9: 5686-92, 2003.
Yokota, T., Milenic, E. D., Whitlow, M. and Schlom, J., Rapid tumor penetration of a single-chain Fv and comparison with other immunoglobulin forms, Cancer Research, 52: 3402-3408, 1992.
You, Y. H., L. J. Hefta, et al. (1998). “Expression, purification, and characterization of a two domain carcinoembryonic antigen minigene (N-A3) in Pichia Pastoris. The essential role of the N-domain.” Anticancer Res. 18: 3193-3202.

What is claimed is:

1. A method for detecting the presence of an analyte in a solution, wherein said analyte comprises at least two mutually exclusive recognition sites that are capable of binding to corresponding mutually exclusive recognition molecules, said method comprising the steps of:
a) providing a first sensor that comprises a first recognition molecule that is capable of binding to one of said recognition sites on said analyte, said first sensor further comprising a first enzyme portion that is attached to said first recognition molecule;
b) providing a second sensor that comprises a second recognition molecule that is capable of binding to another of said recognition sites on said analyte, said second sensor further comprising a second enzyme portion that is attached to said second recognition molecule;
c) mixing said first sensor and said second sensor with a solution that may or may not contain said analyte, wherein said recognition sites on said analyte comprises at least a first recognition site and a second recognition site, said first recognition site being capable of binding to said first sensor and said second recognition site being capable of binding to said second sensor and wherein said first recognition site and second recognition site are located within said analyte such that said first enzyme portion and said second enzyme portion combine to form a biologically active enzyme when said first and second recognition molecules bind to said first and second recognition sites; and
d) detecting if said biologically active enzyme has been formed to thereby provide detection of the presence of said analyte in said solution.

2. A method for detecting the presence of an analyte in a solution according to claim 1 wherein said target analyte is selected from the group consisting of protein, DNA, RNA, lipids and sugars.

3. A method for detecting the presence of an analyte in a solution according to claim 2 wherein said protein is an antigen.

4. A method for detecting the presence of an analyte in a solution according to claim 1 wherein said first and second recognition molecules are selected from the group consisting of single chain antibody fragments, antibody fragments, full antibodies, DNA oligomers, DNA aptamers and PNA oligomers.

5. A method for detecting the presence of an analyte in a solution according to claim 2 wherein said recognition molecule is a single chain antibody fragment, antibody fragment or a full antibody.

6. A method for detecting the presence of an analyte in a solution according to claim 4 wherein said analyte is DNA or RNA and said recognition molecule is a DNA oligomer, DNA aptamer or PNA oligomer.

7. A method for detecting the presence of an analyte in a solution according to claim 1 wherein said biologically active enzyme is selected from the group consisting of renilla luciferase, B-lactamase and B-galactosidase.

8. A method for detecting the presence of an analyte in a solution according to claim 1 wherein said step of detecting if said biologically active enzyme has been formed comprises the steps of reacting said biologically active enzyme with a first reactant to form a first product and detecting the presence of said first product in said solution to thereby provide detection of the presence of said analyte in said solution.

9. A method for detecting the presence of an analyte in a solution according to claim 8 wherein said first product is an activator of a second enzyme and wherein said step of detecting if said biologically active enzyme has been formed comprises the additional steps of:
a) reacting said second enzyme with said first product to form an activated amplifier enzyme;
b) reacting said activated amplifier enzyme with a second reactant to form a second product; and
c) detecting the presence of said second product in said solution to thereby provide detection of the presence of said analyte in said solution.

10. A method for detecting the presence of an analyte according to claim 1 wherein said analyte is located on or in a cell.

11. A method of making an analyte detection system which includes split enzyme biosensors, the method comprising:
a) selecting an analyte with two mutually exclusive recognition sites;
b) selecting two recognition molecules corresponding to the recognition sites; and
c) adding complementary enzyme portions to the two recognition molecules to form said split enzyme biosensors.

12. A method of splitting an enzyme into two functionally complementary portions with reconstituted enzyme activity, the method comprising:
a) splitting the enzyme into two portions;
b) cyclically mutating the two portions;
c) expressing the two portions in the same cell;
d) adding substrate; and
e) selecting cells with reconstituted enzyme activity.

13. A method of making a sensor for a target analyte, the method comprising:
a) choosing a portion of a split enzyme;
b) linking the split enzyme to a target binding domain;
c) randomly mutating the a target binding domain to generate a library; and
d) screening the library for affinity to the target analyte.
